(19) United States
(12) Patent Application Publication
(10) Pub. No.: US 2017/0157000 A1
(43) Pub. Date: Jun. 8, 2017

(54) COSMETIC PRODUCT WITH LIPOSOMAL GROWTH FACTORS

(71) Applicant: DERMOPARTNERS, S.L., Valencia (ES)

(72) Inventors: Gabriel SERRANO SANMIGUEL, Valencia (ES); María NAVARRO MOLINER, Valencia (ES); Ana TORRENS TOMAS, Valencia (ES)

(73) Assignee: DERMOPARTNERS, S.L., Valencia (ES)

(21) Appl. No.: 15/320,497

(22) PCT Filed: Jun. 24, 2014
(86) PCT No.: PCT/ES2014/070513

§ 371 Date: Dec. 20, 2016

(51) Int. Cl. A61K 8/14; A61K 8/97; A61K 9/127; A61Q 19/00; A61K 38/31; A61K 38/19; A61K 8/64; A61Q 19/08

(52) U.S. Cl. CPC A61K 8/14; A61K 8/64; A61K 8/97; A61Q 19/08; A61Q 19/00; A61K 38/31; A61K 38/193; A61K 38/191; A61K 9/127

(57) ABSTRACT

Cosmetic product with liposomated growth factors with properties for repairing and rejuvenating the skin which include in the composition thereof high purity liposomated plant growth factors, liposomated antioxidants, liposomated anti-wrinkle peptides and stem cells in addition to other components for cosmetic use.

### OBJECT OF THE INVENTION

The proposed invention relates to a novel cosmetic product with properties for repairing and rejuvenating the skin which includes in the composition thereof plant growth factors, in addition to antioxidants, anti-wrinkle peptides and stem cells and which is applicable to the sector of beauty, cosmetic and dermatology centers.

### BACKGROUND OF THE INVENTION

Many cosmetic preparations have been known hitherto which have effects for rejuvenating and repairing the skin, but preparations are not known that include liposomes with active ingredients such as high purity plant growth factors together with liposomated antioxidants, anti-wrinkle peptides and stem cells.

### DESCRIPTION OF THE INVENTION

The product which the invention proposes consists of a liposomated preparation in which the liposomes incorporate, as fundamental active ingredients, high purity plant growth factors and antioxidants, in addition to including, without liposomating, anti-wrinkle peptides and stem cells.

The growth factors are natural proteins which stimulate cell growth, proliferation and differentiation. They play an important role in maintaining a structure of healthy skin, upon intervening in the communication between the epidermic and dermic cells by means of bonding to specific receptors on the surface of the cell.

It is well known that the growth factors play an important role for reversing the effects of aging of the skin, the topical application in individuals of growth factors has been demonstrated which acts at a local level and stimulates cell renovation, the reduction of wrinkles and the increase of collagen synthesis.

The main advantage of the product of the present invention is that it concerns high purity plant growth factors which, as they are incorporated in liposomes, their penetration through the skin and their stability is improved while avoiding their degradation like proteins that they are.

Among the plant growth factors included in the composition, there are the factors: HGH (human growth hormone), Nicotiana benthamiana Sh-Polypeptide-7, GM-CSF (Granulocyte Macrophage Colony-Stimulating Factor) Nicotiana benthamiana Sh-Polypeptide-45 and TGFb2 (Tumor growth factor beta 2) Nicotiana benthamiana Hexapeptide-40 Sh-Polypeptide-76.

Nicotiana benthamiana Sh-Polypeptide-7 is a single chain recombinant human peptide, produced in a transitory manner in the expression of Nicotiana benthamiana in plants. The starting gene is a copy synthesized from the human gene which codes for the growth hormone (GH) used as such or adapted to the host. It contains a maximum of 197 amino acids. The protein consists of the appropriate sequence of the 20 standard amino acids.

Nicotiana benthamiana Sh-Polypeptide-45 is a single chain of recombinant human peptide, produced in a transitory manner in the expression of Nicotiana benthamiana in plants. The starting gene is a synthetic copy of the human gene which codes for the factor for stimulating colonies of granulocytes and macrophages used as such or adapted for the production of the host. It contains a maximum of 127 amino acids which may contain disulfide bonds and/or glycosylation. The protein consists of the appropriate sequence of the 20 standard amino acids.

Nicotiana benthamiana Hexapeptide-40 sh-Polypeptide-76 contains two identical polypeptide chains joined by a single disulfide bond. They are produced in a transitory manner in the expression of Nicotiana benthamiana in plants. The starting gene is a synthetic copy of the human gene which codes for the transforming beta 2 growth factor used as such or adapted for the production of the host. It contains a maximum of 237 amino acids which may contain disulfide bonds and/or glycosylation. The protein consists of the appropriate sequence of the 20 standard amino acids.

In addition, among the liposomated antioxidants which are included in the composition there are ergothioneine, quercetin and pterostilbene and among the anti-wrinkle peptides, also liposomated, there are Centella asiatica extract, palmitoyl tripeptide-3 and caprooyl tetrapeptide-3.

Lastly, the product also incorporates as the active ingredient, without liposomating, extracts of stem cells of Malus domestica.

In the tests carried out, the data of which is stated below, it is checked that these growth factors in the form of liposomes maintain the same activity as the factor in free form, but their penetration through the skin and their stability is improved.

### PREFERRED EMBODIMENT OF THE INVENTION

As a preferred embodiment, a liposomated cosmetic product is proposed which has different cosmetic forms such as nutritive cream, solution, serum, emulsion, suspension, etc. in which the different components may be found in the case of the cream in the following proportions of active ingredients and of liposomes:

| INCI DESIGNATION | % | % LIPOSOMES |
|---|---|---|
| WATER | 50-75 | |
| CAPRYLIC/CAPRIC | 1-5 | |
| TRIGLYCERIDE | | |
| SHEA BUTTER EXTRACT | 1-5 | |
| PROPANEDIOL | 1-5 | |
| SWEET ALMOND OIL | 1-5 | |
| DIMETICONE | 1-5 | |
| CETYL ALCOHOL | 1-5 | |
| GLYCERYL STEARATE | 1-5 | |
| LECITHIN | 1-5 | |
| CETEARYL ALCOHOL | 0.1-1 | |
| TOCOPHERYL ACETATE | 0.1-1 | |
| ALCOHOL | 0.1-1 | |
| PEG-75 STEARATE | 0.1-1 | |
| PHENOXYETHANOL | 0.1-1 | |
| POLYSORBATE 20 | 0.1-1 | |
| CETETH-20 | 0.1-1 | |
| STEARETH-20 | 0.1-1 | |
| XANTHAN GUM | 0.1-1 | |
| TRIETHANOLAMINE | 0.1-1 | |
| ACRYLATES/C10-30 ALKYL | 0.1-1 | |
| ACRYLATE CROSSPOL | | |
| ETHYLHEXYLGLYCERIN | 0.1-1 | |
| SODIUM CHLORIDE | 0.1-1 | |
| MALUS DOMESTICA STEM CELL | 0-0.1 | |
| EXTRACT | | |
| GLYCERIN | 0-0.1 | |
| DISODIUM EDTA | 0-0.1 | |
| PERFUME | 0-0.1 | |
| SODIUM CHOLATE | 0-0.1 | |
| PTEROSTILBENE | 0-0.1 | LIP. PTEROSTILBENE 1% |
| CENTELLA ASIATICA EXTRACT | 0-0.1 | LIP. CENTELLA ASIATICA |
| | | EXTRACT 1.25% |
| PALMITOYL TRIPEPTIDE-3 | 0-0.1 | LIP. PALMITOYL TRIPEPTIDE -3 3.5% |
| CAPROOYL TETRAPEPTIDE-3 | 0-0.1 | LIP. CAPROOYL TETRAPEPTIDE -3 1.25% |
| QUERCETIN | 0-0.1 | LIP. QUERCETIN 1% |
| DEXTRAN | 0-0.1 | |
| TROMETHAMINE | 0-0.1 | |
| HYDROCHLORIC ACID | 0-0.1 | |
| ERGOTHIONEINE | 0-0.1 | LIP. ERGOTHIONEINE 1% |
| DIPOTASSIUM PHOSPHATE | 0-0.1 | |
| POTASSIUM PHOSPHATE | 0-0.1 | |
| NICOTIANA BETHANIANA SH- | 0-0.1 | LIP. NICOTIANA BETHANIANA |
| POLYPEPTIDE-7 | | SH-POLYPEPTIDE-7 5% |
| NICOTIANA BENTHAMIANA | 0-0.1 | LIP. NICOTIANA BENTHAMIANA |
| HEXAPEPTIDE-40 SH- | | HEXAPEPTIDE-40 SH- |
| POLYPEPTIDE-76 | | POLYPEPTIDE-76 5% |
| NICOTIANA BETHAMIANA SH- | 0-0.1 | LIP. NICOTIANA BETHAMIANA |
| POLYPEPTIDE-45 | | SH-POLYPEPTIDE-45 5% |

Another preferred embodiment is in the case of the serum where the different ingredients are found in the following proportions:

| INCI DESIGNATION | % | % LIPOSOMES |
|---|---|---|
| WATER | 75-100 | |
| GLYCERIN | 5-10 | |
| BIS-PEG-18 METHYL ETHER | 1-5 | |
| DIMETHYL SILANE | | |
| PROPANEDIOL | 1-5 | |
| LECITHIN | 1-5 | |
| ALCOHOL | 1-5 | |
| HYDROGENATED CASTER OIL | 0.1-1 | |
| PEG-40 | | |
| PHENOXYETHANOL | 0.1-1 | |
| POLYSORBATE 20 | 0.1-1 | |
| TOCOPHERYL ACETATE | 0.1-1 | |
| TRIETHANOLAMINE | 0.1-1 | |
| ACRYLATES/C10-30 ALKYL | 0.1-1 | |
| ACRYLATE CROSSPOL | | |
| DISODIUM EDTA | 0.1-1 | |
| ETHYLHEXYLGLYCERIN | 0.1-1 | |
| SODIUM CHLORIDE | 0.1-1 | |
| PERFUME | 0.1-1 | |
| MALUS DOMESTICA STEM CELL | 0.1-1 | |
| EXTRACT | | |
| SODIUM CHOLATE | 0.1-1 | |
| PTEROSTILBENE | 0.1-1 | LIP. PTEROSTILBENE 1% |
| XANTHAN GUM | 0.1-1 | |
| CENTELLA ASIATICA EXTRACT | 0.1-1 | LIP. CENTELLA ASIATICA |
| | | EXTRACT 1.25% |
| PALMITOYL TRIPEPTIDE-3 | 0.1-1 | LIP. PALMITOYL TRIPEPTIDE -3 3.5% |
| CAPROOYL TETRAPEPTIDE-3 | 0-0.1 | LIP. CAPROOYL TETRAPEPTIDE -3 1.25% |
| QUERCETIN | 0-0.1 | LIP. QUERCETIN 1% |
| DEXTRAN | 0-0.1 | |
| TROMETHAMINE | 0-0.1 | |
| HYDROCHLORIC ACID | 0-0.1 | |
| ERGOTHIONEINE | 0-0.1 | LIP. ERGOTHIONEINE 1% |
| DIPOTASSIUM PHOSPHATE | 0-0.1 | |
| POTASSIUM PHOSPHATE | 0-0.1 | |
| NICOTIANA BENTHAMIANA | 0-0.1 | LIP. NICOTIANA BENTHAMIANA |
| HEXAPEPTIDE-40 SH- | | HEXAPEPTIDE-40 SH- |
| POLYPEPTIDE-76 | | POLYPEPTIDE-76 8% |
| NICOTIANA BETHANIANA SH- | 0-0.1 | LIP. NICOTIANA BETHANIANA |
| POLYPEPTIDE-7 | | SH-POLYPEPTIDE-7 5% |
| NICOTIANA BETHAMIANA SH- | 0-0.1 | LIP. NICOTIANA BETHAMIANA |
| POLYPEPTIDE-45 | | SH-POLYPEPTIDE-45 5% |

### Clinical Studies and Description of the Drawings

### Analysis of the Activity of the Free Factor Versus the Factor Encapsulated in Fibroblasts of Skin and Human Keratinocytes

Factors analyzed:
Factors GM-CSF (Granulocyte Macrophage Colony-Stimulating Factor), HGH (human growth hormone), TGFb2 (Tumor growth factor beta 2) and follistatin.
Factors GM-CSF, HGH, TGFb2 and follistatin encapsulated in liposomes.
GM-CSF and HGH→50 mg/ml lipids, 400 ng/ml factor
TGFb2 and follistatin→50 mg/ml lipids, 200 ng/ml factor
   Methods used:
Cell proliferation
Collagen elastin/synthesis by means of quantitative PCR
Migration
Anti-inflammatory effect by means of quantitative PCR

### MTT Cell Proliferation Assay

This assay is based on metabolic reduction of bromide 3-(4,5-dimethylthiazol-2-yl)-2,5-diphenyltetrazolium (MTT) carried out by the mitochondrial succinate dehydrogenase enzyme in a blue colored compound (formazan), allowing the mitochondrial functionality of the treated cells to be determined. This method has been very widely used to measure cell survival and proliferation.

The quantity of live cells is proportional to the quantity of formazan produced. This method was developed by Mosmann in 1983, modified in 1986 by Francois Denizot and Rita Lang.

### Methodology

1. Using trypsin, separate the cells and re-suspend them in supplemented culture medium (10% of bovine fetal serum and antibiotic).
2. Determine the number of cells required per well using a Neubauer camera (HaCaT: 5,000 cells/96 well, 30,000 cells/24 well; HSF: 4,000 cells/96 well, 30,000 cells/24 well.
3. Incubate at 37° C. and 5% of CO2 for 24 hours to allow for the adherence thereof.
4. Prepare the mixtures of free and liposomated factor at the indicated concentrations, remove the medium and add the mixtures.
5. Incubate at 37° C. and 5% of CO2 for 72 hours.
6. Add MTT (1.9 mg/ml) in PBS solution (25 μl total) over the medium
7. Incubate for 3 hours at 37° C. to allow for the formation of formazan crystals.
8. Then remove the supernatant and add 100 μl of DMSO.
9. Incubate for 30 minutes at 37° C. to allow for the formazan crystals to dissolve
10. The optical density reading (OD) is carried out in a spectrophotometer at a wavelength of 570 nm.
   The viability percentage is obtained in the following manner:

% viability—DO cells treated×100/DO control cells

### Wound Healing Assay (Cell Migration)

The objective of the wound healing assay is the study of cell migration. It is based on observing the behavior of a confluent cell monolayer on which a hole or wound has been previously made. The cells at the border of the hole move towards the opening until new cell-cell contacts are established, thus closing the wound.

The basic steps involve creating the wound or cell free area on the cell monolayer, the capture of images periodically during the experiment and the comparison of all the images to determine the velocity of cell migration.

### Methodology

1. Cultivate the cells until confluence (100% approx.) in plates of 6 wells (HaCaT: 500,000 cells/6 well; HSF: 500,000 cells/6 well)
2. Incubate at 37° C. and 5% of CO₂ for 24 hours.
3. Scaling of the wounds (2 wounds per horizontal trajectory well using yellow sterile points (P200). They are dragged along the monolayer with an angle of inclination of 30 degrees (not perpendicular to the plate) and guided with a previously marked section. Mark three points for taking measurements.
4. Leave to rest for 15 minutes before adding the treatment.
5. Prepare the mixtures of free and liposomated factors at a concentration of 1 ng/ml, the medium is removed and add the mixtures.
6. Incubate at 37° C. and 5% of CO₂ for 72 hours.
7. Take measurements at the times 0, 24, 48 and 72 hours.

The percentage of migration is obtained in the following manner:

% migration=(time measured 0−time measured X/time measured 0)*100

### Gene Expression Analysis by Means of PCR in Real Time. Elastin Expression Assay and Proinflammatory Cytokines

PCR in real time or quantitative PCR is a variation of standard PCR used for quantifying DNA or messenger RNA (mRNA) of a sample. Using specific sequence primers, it is possible to determine the number of copies or the relative quantity of a determined DNA or RNA sequence.

When PCR in real time is combined with a retro-transcription or RT reaction (RTPCR), the mRNA quantity of a sample may be determined by means of a relative quantification. Said quantification is termed relative if the relative quantity or ratio of mRNA of a specific gene is compared with respect to the mRNA quantity of a constitutive gene (endogenous control, GAPDH in this case) among different samples. This is what is termed as normalization of the specific gene expression or normalizing with respect to the different total concentration of RNA of the samples since if the quantity of endogenous control varies it is due to changes in the total RNA quantity used in the synthesis of cDNA, not to changes in its expression. For the quantification, the quantity of amplicon produced is measured in each cycle of PCR. The quantification of the product is produced by means of the addition of fluorophores which join to the amplicon in a quantitative manner such that the greater the product, the greater the fluorescence that will be emitted.

The quantification method which has been used is ΔΔCt in which the Cts of the tested gene and reference gene (ΔCt) are compared directly in each sample and subsequently the (ΔCt) of the experimental samples are compared with respect to the control sample, in order to apply said method it is necessary for the efficiencies of both genes to be similar.

### Methodology

1. Using trypsin, separate the cells and re-suspend them in supplemented culture medium (10% of bovine fetal serum and antibiotic).
2. Determine the number of cells required per well using a Neubauer camera (HaCaT: 9,000 cells/96 well; HSF: 9,000 cells/96 well)
3. Incubate at 37° C. and 5% of CO2 for 24 hours to allow for the adherence thereof.
4. Prepare the mixtures of free and liposomated factor at the indicated concentrations, remove the medium and add the mixtures.
5. Incubate at 37° C. and 5% of CO2 for 72 hours.
6. RNA extraction using the kit: E.Z.N.A.® Total RNA (Omega Bio-Tek, Inc., United States) following the manufacturer instructions. The isolated RNA was kept at −80° C. until its use. A microgram of RNA, estimated by the spectrophotometry NanoDrop 200 c (Thermo FisherScientific Inc.) was used for obtaining cDNA using M-MLV reverse transcriptase (Invitrogen) according to the traditional procedures.
7. The PCRq assays were carried out using the ABI PRISM 7300 system (Applied Biosystems, NJ, USA) and SYBR green. The reactions were carried out in a volume of 20 μl in which there was 2 μl of cDNA, 900 nm of each primer, 200 nm of probe and 10 μl of the TaqMan universal mix master (Applied Biosystems) or SYBR green. The conditions of the cycles were 50° C. for 2 minutes and 95° C. for 10 mins, followed by 40 cycles at 95° C. for 15 seconds and 60° C. for 1 minute. The expression was analyzed by the method of 2-DCt where ΔCt is determined by subtracting the value of ΔCt from the GAPDH gene, used as the endogenous control, at the value of the objective Ct.

### Results

1. Cell Proliferation

1.1 Cell Proliferation with HGH

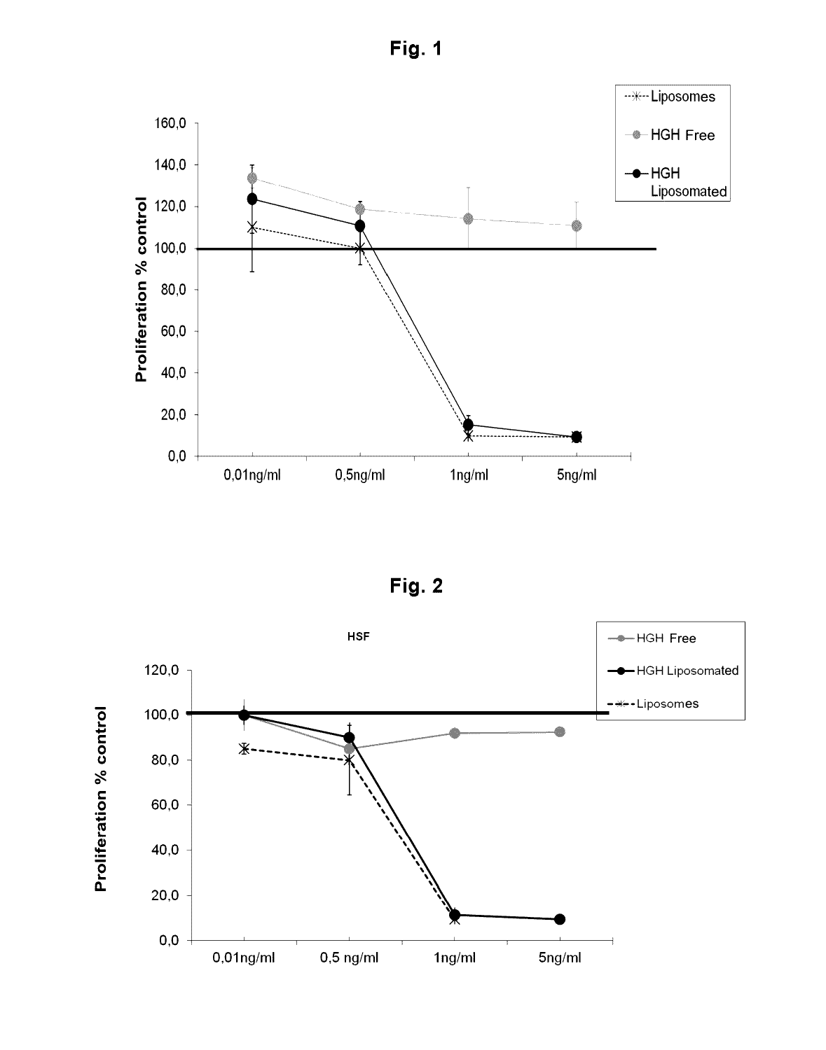

In FIG. 1, it may be observed that in human keratinocytes, both the free and the liposomated factor generate cell proliferation of 20-40% above the untreated cells (which indicate 100% proliferation) at low concentrations (0.01 ng/ml-0.5 ng/ml). However, at concentrations greater than 1 ng/ml, a high decrease of the cell viability is observed with the liposomated factor, but not with the free factor which maintains the cell proliferation around 20% above the untreated cells. In addition, the liposomes do not induce proliferation at low concentrations (0.01 ng/ml-0.5 ng/ml), although they do induce a high decrease of the cell viability at concentrations greater than or equal to 1 ng/ml.

In addition in FIG. 2, it may be observed that in human fibroblasts, the free factor does not generate proliferation, being maintained around 100%. However, at concentrations greater than 1 ng/ml, a high decrease of the cell viability is observed with the liposomated factor, but not with the free factor which is maintained around 100%. In addition, the liposomes induce a decrease of the cell viability (20%) at low concentrations (0.01 ng/ml-0.5 ng/ml) and a high decrease of the cell viability at concentrations greater than or equal to 1 ng/ml.

1.2 Cell Proliferation with GM-CSF

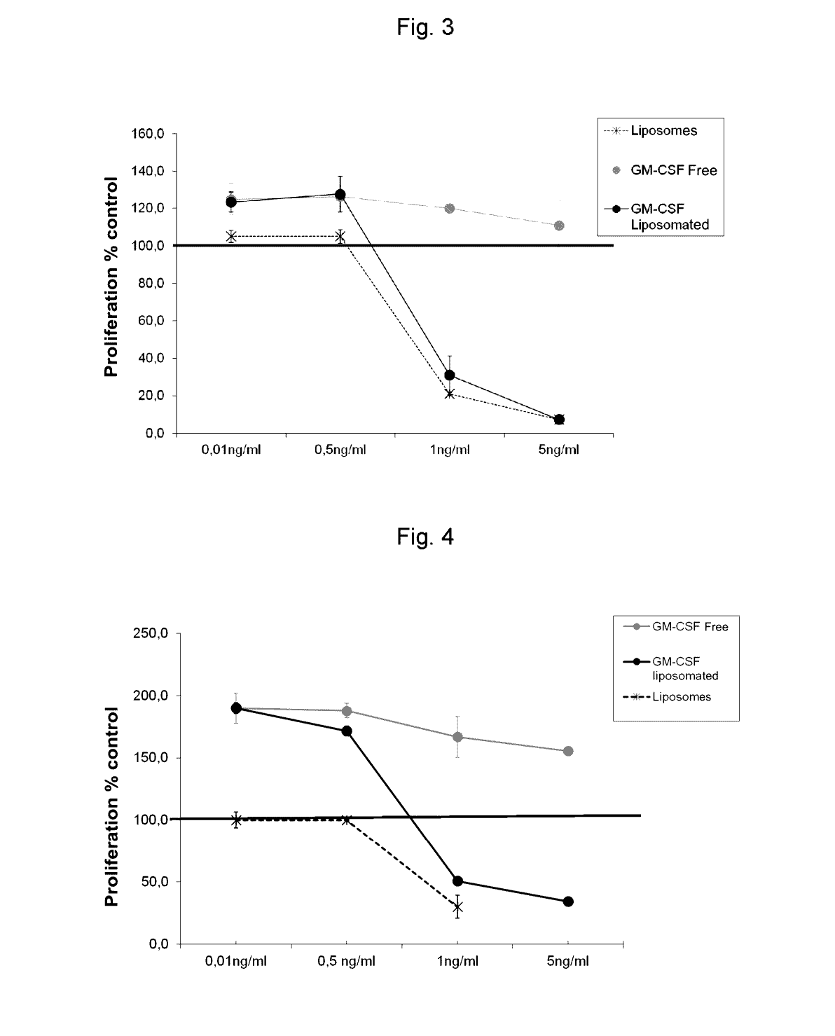

In FIG. 3, it may be observed that in human keratinocytes, both the free and the liposomated factor generate cell proliferation of 20% above the untreated cells (which indicate 100% proliferation) at low concentrations (0.01 ng/ml-0.5 ng/ml). However, at concentrations greater than 1 ng/ml, a high decrease of the cell viability is observed with the liposomated factor, but not with the free factor which maintains the cell proliferation around 20% above the untreated cells. In addition, the liposomes do not induce proliferation at low concentrations (0.01 ng/ml-0.5 ng/ml), although they do induce a high decrease of the cell viability at concentrations greater than or equal to 1 ng/ml.

In addition in FIG. 4, it may be observed that in human fibroblasts, the free factor and the liposomated factor generate cell proliferation of 100% above the untreated cells (which indicate 100% proliferation) at low concentrations (0.01 ng/ml-0.5 ng/ml). However, at concentrations greater than 1 ng/ml, a decrease of the cell viability of 50-60% is observed with the liposomated factor, but not with the free factor which maintains cell proliferation around 60% above the untreated cells. In addition, the liposomes do not induce proliferation at low concentrations (0.01 ng/ml-0.5 ng/ml), although they do induce a decrease of the cell viability of 50-60% at concentrations greater than or equal to 1 ng/ml.

1.3 Cell Proliferation with Follistatin

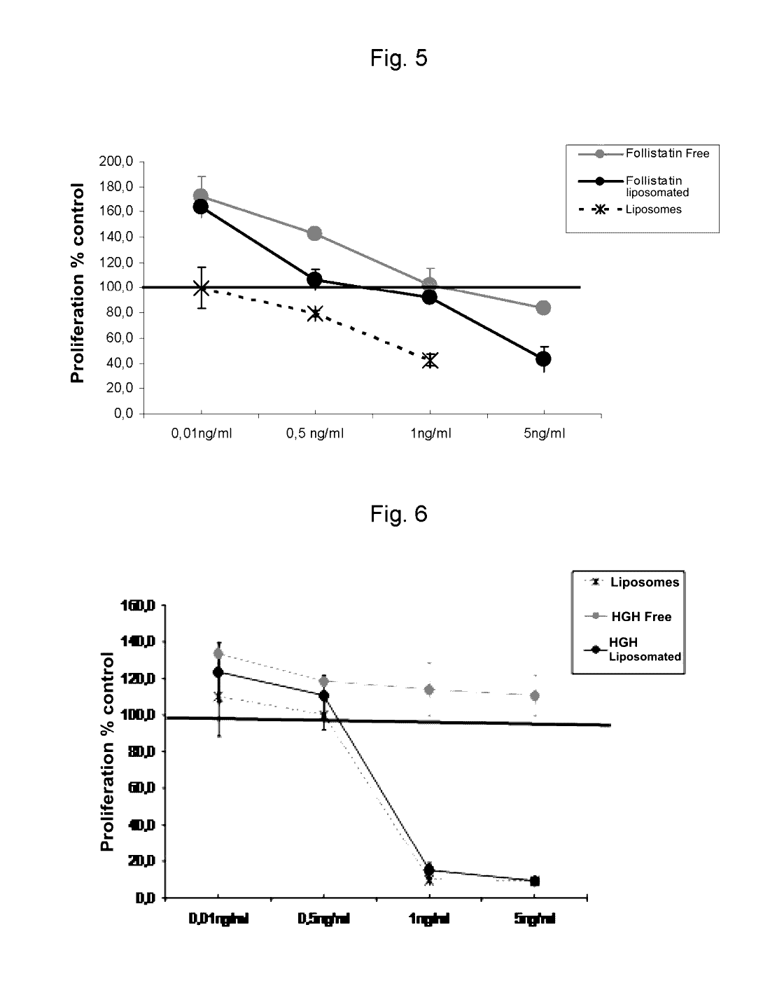

In FIG. 5, it may be observed that in human fibroblasts, both the free factor and the liposomated factor generate cell proliferation of 60-70% above the untreated cells (which indicate 100% proliferation) at low concentrations (0.01 ng/ml). However, at a concentration of 0.5 ng/ml, the free factor generates a proliferation of 40%, while the liposomated factor does not generate proliferation. In addition, at concentrations greater than 1 ng/ml, a decrease of the cell viability of 50% is observed with the liposomated factor, but not with the free factor which maintains cell proliferation around the control. In addition, the liposomes do not induce proliferation at low concentrations (0.01 ng/ml-0.5 ng/ml), although they do induce a decrease of the cell viability at concentrations greater than 0.5 ng/ml.

2. Cell Migration (Wound Healing)

2.1 Cell Migration with HGH

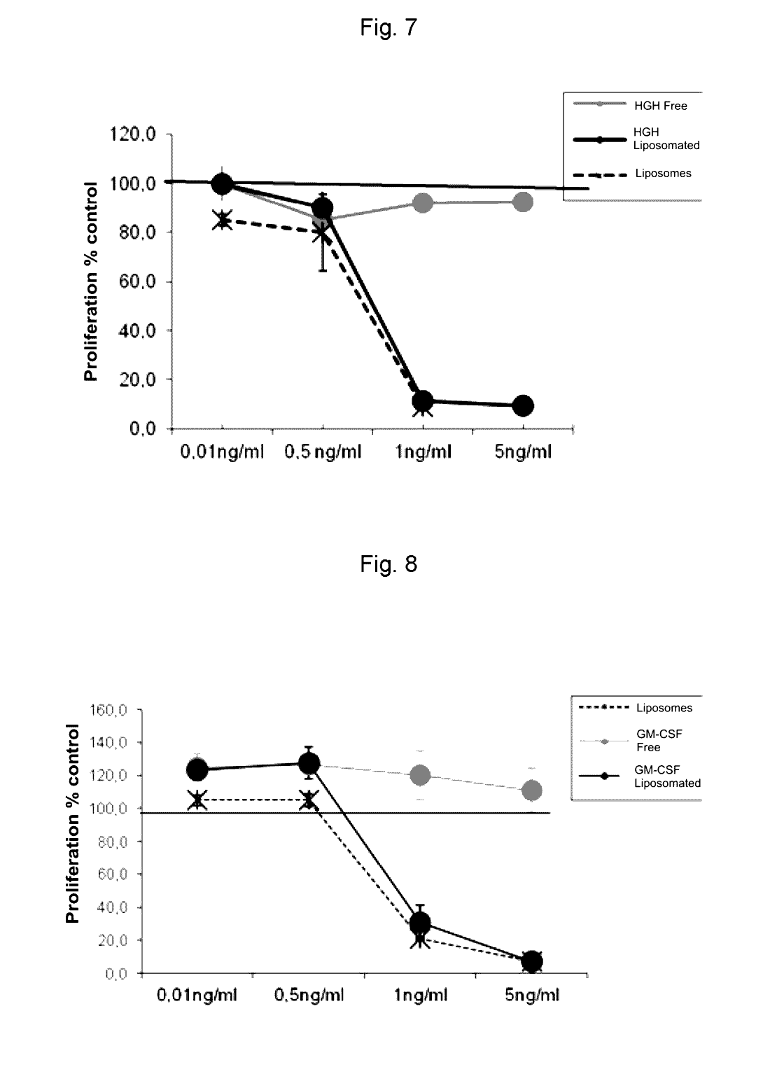

In FIG. 6, a rapid repair velocity of the wound is observed with the free factor in human keratinocytes since at 24 hours of treatment, the wound had closed 40% more quickly than in the control cells and at 48 hours it was already completely closed. However, it is observed that the liposomated factor makes it difficult to close the wound, it being closed more slowly than in the control cells. However, in FIG. 7, it is observed that in human fibroblasts there is hardly any difference seen in the repair velocity of the wound among the cells treated with free factor and the control cells.

2.2. Cell Migration with GM-CSF

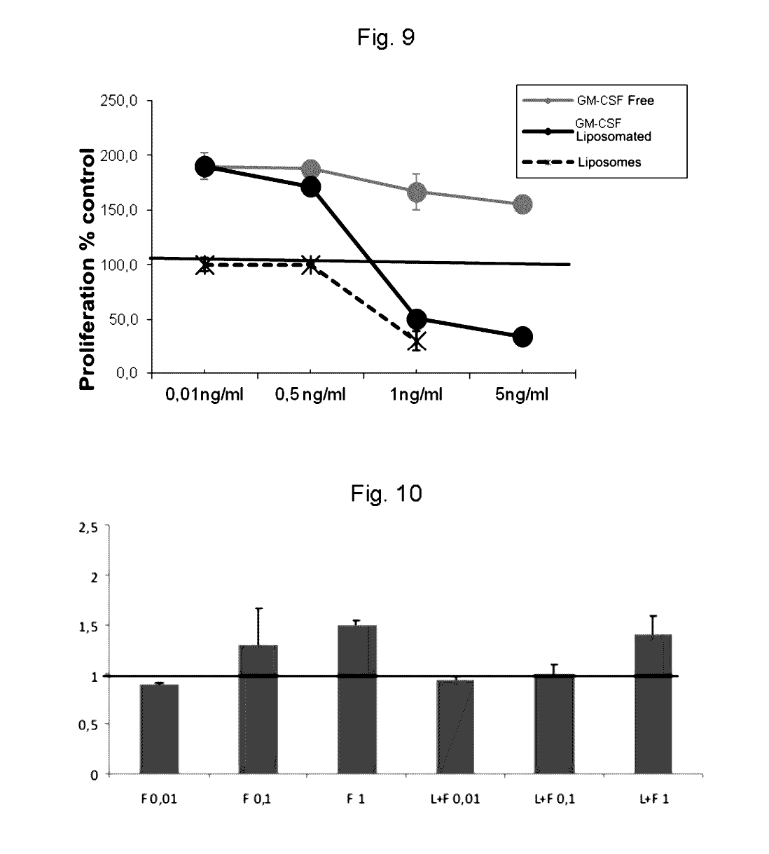

In FIG. 8, a slight increase in the repair velocity of the wound is observed in human keratinocytes with respect to the control cells since at 24 hours of the treatment the wound had closed 13% more quickly than the control and at 48 hours it was already completely closed. However, it is observed that the liposomated factor makes it difficult to close the wound, it being closed more slowly than in the control cells. In addition, in FIG. 9 it is observed that in human fibroblasts the cells treated with free factor show greater repair velocity of the wound, 20% more quickly with respect to the control. However, like the other cases, the liposomated factor makes it difficult to close the wound, it being closed more slowly than in the control cells.

It should be highlighted that the results obtained with the migration assays are similar to those obtained in the cell proliferation assays and therefore they corroborate them.

3. Induction of Elastin Synthesis by Means of Measuring Transcripts of the Gene of the Elastin by Quantitative PCR

3.1 Induction of Elastic Synthesis by HGH

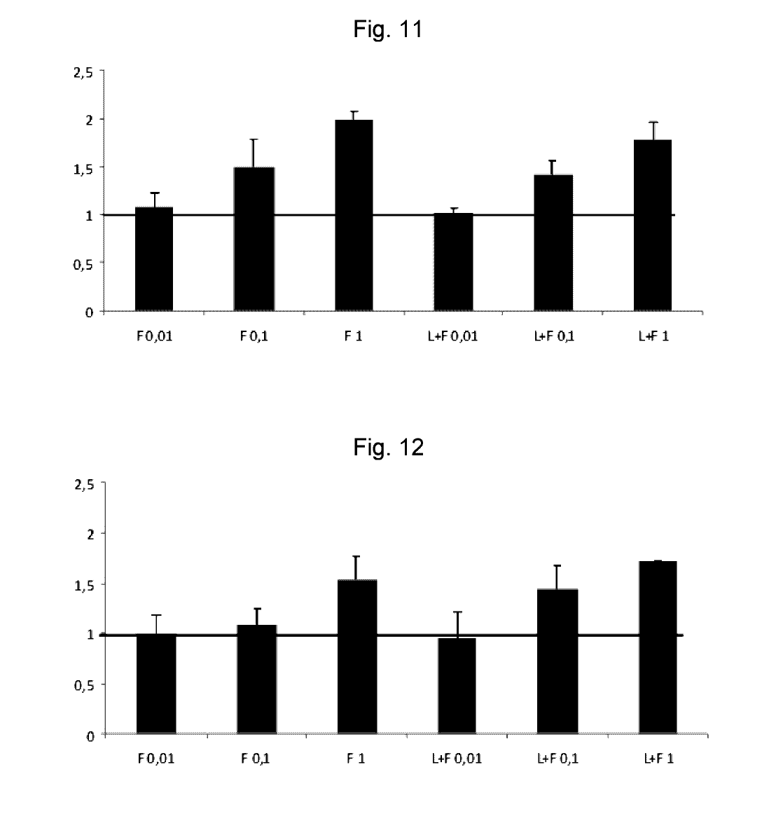

In FIGS. 10 and 11, an increase in the synthesis of elastin is observed in the cells treated both with the free factor and with the liposomated factor with respect to the control cells (indicated by the horizontal line=value 1), both in fibroblasts and in keratinocytes. This increase is dose dependent, increasing as the concentration of the factor is increased.

3.2 Induction of Elastin Synthesis by GM-CSF

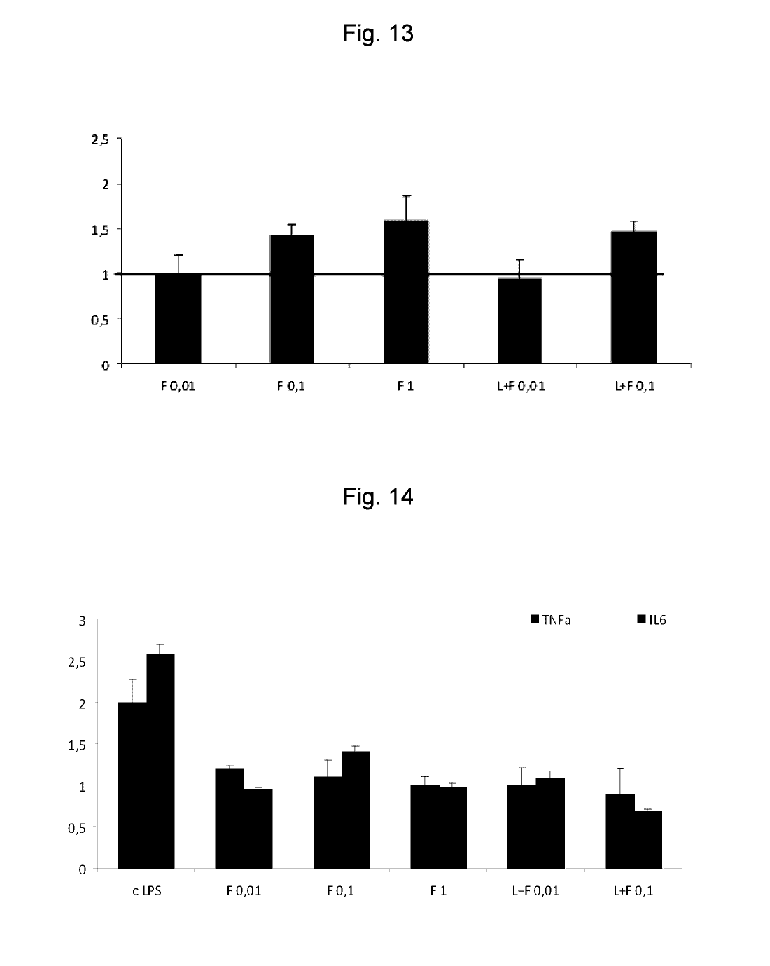

In FIGS. 12 and 13, an increase in the synthesis of elastin is observed in the cells treated both with the free factor and with the liposomated factor with respect to the control cells (indicated by the horizontal line=value 1), both in fibroblasts and in keratinocytes. This increase is dose dependent, increasing as the concentration of the factor is increased.

4. Analysis of the Anti-Inflammatory Effect by Means of Measuring Transcripts of the Anti-Inflammatory Genes IL6 and TNFa BY QUANTITATIVE PCR

In this assay on the cells, a mild inflammation was induced on them with LPS prior to the treatment. To this end, medium with 200 ng/ml LPS was added to them, for three hours, it was removed and the medium with the factors was added.

4.1 Analysis of the Anti-Inflammatory Effect Induced by HGH

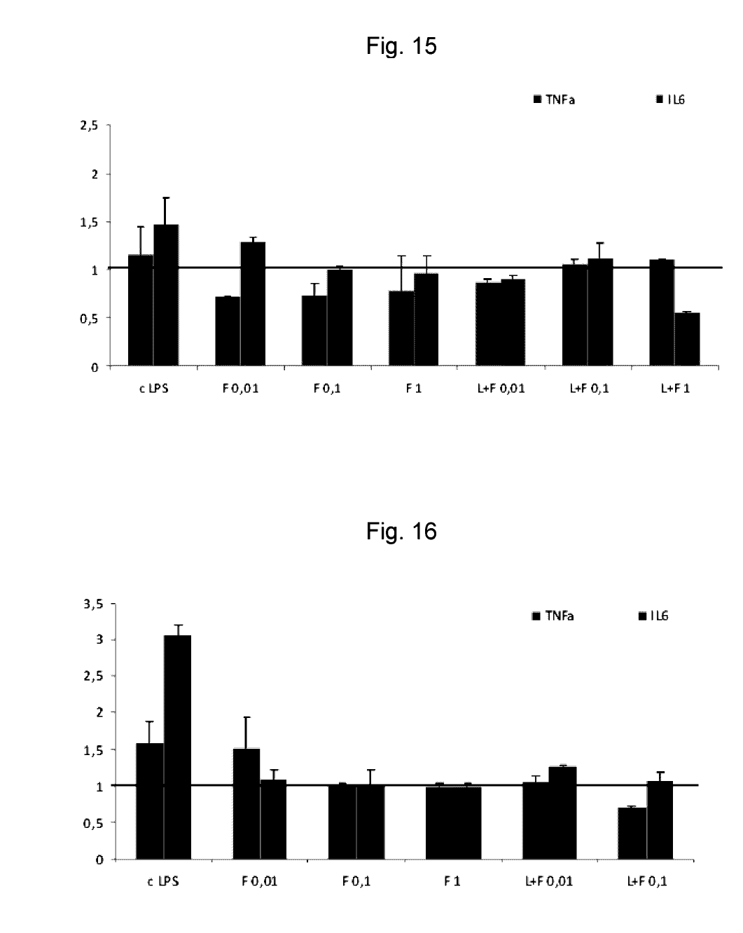

In the FIGS. 14 and 15, a decrease of the inflammation is observed, that is to say, the transcript levels of TNFa and IL6 in the cells treated with the free factor or with the liposomated factor after prior inflammation with LPS (LPS c bars) having been induced in them. These results indicate the anti-inflammatory effect of the HGH factor, both free and liposomated. This decrease is dose dependent, increasing as the concentration of the factor is increased.

4.2 Analysis of the Anti-Inflammatory Effect Induced by GM-CSF

In FIG. 16, a decrease of the inflammation is observed, that is to say, the transcript levels of TNFa and IL6 in the cells treated with the free factor or with the liposomated factor after prior inflammation with LPS (LPS c bars) having been induced in them. These results indicate the anti-inflammatory effect of the GM-CSF factor, both free and liposomated. This decrease is dose dependent, increasing as the concentration of the factor is increased.

With the nature of the present invention sufficiently described, all that remains to be added is that said invention may undergo certain variations in terms of components and percentages, provided that said alterations do not substantially vary the characteristics which are claimed below.

1. A cosmetic product with liposomated growth factors which comprises:
an active ingredient which consists of a mixture of liposomes which contain plant growth factors HGH, GM-CSF and TGFb2;
a second active ingredient which consists of a mixture of liposomes which incorporate in their interior antioxidants such as ergothioneine, quercetin and pterostilbene;
a third active ingredient consists of a mixture of liposomes of anti-wrinkle peptides; and
a fourth active ingredient consists of stem cell extract with liposomating.

2. The cosmetic product with liposomated growth factors according to claim 1, in which each one of the growth factors, antioxidants and anti-wrinkle peptides are separately encapsulated in liposomes.

3. The cosmetic product with liposomated growth factors according to claim 1, in which the growth factors included in the liposomes have a purity greater than 95%.

4. The cosmetic product with liposomated growth factors according to claim 1, in which the liposomes with growth factors are present at percentages of the following:
HGH liposomes (human growth hormone) Nicotiana benthamiana Sh-Polypeptide-7 of 4 to 10%;
GM-CSF liposomes (Granulocyte Macrophage Colony-Stimulating Factor) Nicotiana benthamiana Sh-Polypeptide-45 of 2 to 7%; and
TGFb2 liposomes (Tumor growth factor beta 2) Nicotiana benthamiana Hexapeptide-40 Sh-Polypeptide-76 of 2 to 10%.

5. The cosmetic product with liposomated growth factors according to claim 1, in which the liposomes with antioxidants in the interior are at a percentage of 1 to 3%.

6. The cosmetic product with liposomated growth factors according to claim 1, in which the anti-wrinkle peptides are palmitoyl tripeptide-3, caprooyl tetrapeptide-3 and centella asiatica extract.

7. The cosmetic product with liposomated growth factors according to claim 1, in which the liposomes with anti-wrinkle peptides are at a percentage of 1 to 4.5%.

8. The cosmetic product with liposomated growth factors according to claim 1, in which the stem cell extract is Malus domestica stem cell extract is 0.1 to 1.5%.

9. The cosmetic product with liposomated growth factors according to claim 8, in which the stem cell extract is at a percentage of 0.1 to 1.5%.
